(19) United States
(12) Patent Application Publication
(10) Pub. No.: US 2023/0293810 A1
(43) Pub. Date: Sep. 21, 2023

(54) INFUSION UNITS AND RELATED METHODS

(71) Applicant: INFUZAMED LLC, Claremont, CA (US)

(72) Inventor: Aamir Zain Jamal, Claremont, CA (US)

(21) Appl. No.: 18/324,392

(22) Filed: May 26, 2023

Related U.S. Application Data
(63) Continuation-in-part of application No. 17/739,589, filed on May 9, 2022.
Application No. 17/739,589 is a continuation-in-part of application No. 16/913,486, filed on Jun. 26, 2020, now Pat. No. 11,364,339.

(51) Int. Cl. A61M 5/142; A61M 5/172

(52) U.S. Cl. CPC A61M 5/14244; A61M 5/1723; A61M 2205/3569; A61M 2205/3331

(57) ABSTRACT

Improvements in an infusion unit are disclosed. The infusion unit is a portable pump that can be used with patient that have wrist access or decubital PICC/MID-LINES without the risk of line accidently getting pulled. The infusion unit is in the shape of a convenient shell that has an appearance of an arm cast. There are no hoses that extend beyond the body of the infusion unit and any vein penetration is completely covered under the body of the infusion unit. The secure enclosed unit prevents unauthorized tampering and entry into the unit. The medication dosing can be remotely monitored, fusion resumed, changed by nursing and the patient. The infusion unit may include a wireless connection to a network to adjust medication and monitor vital signs, namely heart rate, O2 level and blood pressure that can be monitored and recorded for review.

### CROSS-REFERENCE TO RELATED APPLICATIONS

This application is a continuation-in-part of U.S. Nonprovisional patent application Ser. No. 17/739,589, filed May 9, 2022; which is a 371 U.S. National Stage Application of International PCT application PCT/US20/41542, filed on Jul. 10, 2020; which is a continuation-in-part of U.S. Nonprovisional patent application Ser. No. 16/913,486, filed on Jun. 26, 2020, now issued as U.S. Pat. No. 11,364,339 on Jun. 21, 2022; which claims the priority benefit of U.S. Provisional Patent Application Ser. No. 62/873,109, filed Jul. 11, 2019; the contents of each of which are herein incorporated by reference in their entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates to improvements in technologies used to infuse drugs into a person. More particularly, the present infusion unit is a self-contained infusion unit including drugs and a power supply that are secured around the arm of a user, in which a line is inserted into the vein of a person so the drugs can be infused into the person.

### BACKGROUND

At some point in most people's lives, they will require medical attention in which an intravenous line is inserted into a vein of a person, and drugs, nutrition, or medication are slowly infused into the person. All antibiotics, saline, and other fluids are given to the patients to directly introduce fluid or medication into the blood stream to effect more prompt and effective responses. Pumps are used in the hospital where nurses will monitor and give medications via pole hanging bags of medications and large pumps for infusion. Sending a patient home requires one or more nurses to go to the patient's home two to three times a day to give medications intravenously into the wrist of a patient or through a peripherally inserted central catheter or PICC line. The most common method is to hang a bag containing the medication and infuse the drugs through a long tube. This method can cause several problems for mobility of the person. Long tubing lengths may pose problems such as accidental intravenous-line pull, infiltration, entanglement of tubing, and/or tubing kinking. Replacing drugs requires a physician or other medical personnel to properly and safely make changes. For these reasons, infusion units are mostly used within a medical facility. Even within a hospital, the mobility of a person can be a problem when a patient needs to exit a bed to use a bathroom or walk down a hallway.

Thus, there is a need new and useful methods and devices for the infusion of medication into a patient to increase comfort, increase mobility, and/or reduce technical issues.

### SUMMARY

It is an object of the infusion unit to be a portable pump that can be used with patient that have wrist access or decubital PICC/MID-LINES without the risk of line accidently getting pulled due to the proximity of the infusion unit. The infusion unit is in the shape of a conveniently shell that has an appearance of an arm cast. The integrated unit does not have external wiring or hoses between the infusion unit and the patient. The shell is also constructed to reduce rotation of the infusion unit on an arm where rotation can result in harm to the vein and can further damage internal medication and the operating mechanism. The unit is wrapped around the forearm and locked to the thumb holds it at a place. Unit is structured in such a way that the hardware is deep in the unit with cover to avoid accidental damage to the unit by water or electromagnetic or other element exposure to some extent. Material is such that it does not fracture with simple trauma to the infusion unit.

It is an object of the infusion unit to provide tightly regulated medication dosing that reduces the chance of overdosing and/or underdosing. Accuracy-ensured dosing is provided with micro infusions (e.g., delivered by stepper motor actuation), such as Antibiotics and NS or Riger-lactate or TPN insulin and other replacement hormones. Due to dosing being administered with wireless controls and with real-time monitoring of the infusion unit, human error is virtually eliminated.

It is another object of the infusion unit to be used on out-patients where there is no need for a medication pole that can easily wrap around the body of the user the chance of accidentally pulling the IV-line from the arm is removed. There are no hoses that extend beyond the body of the infusion unit and any vein penetration is completely covered under the body of the infusion unit, minimizing line infiltration.

It is another object of the infusion unit to have a secure enclosed unit that has a secure enclosure to prevent unauthorized tampering and entry into the unit. Security of the internal medication is important when the infusion unit is not in a medical facility. The infusion unit can detect tampering and can notify authorities if the infusion unit is opened.

It is another object of the infusion unit to deliver the medication with a stepper motor for antibiotics of about 50 mL or larger or an infusion over a 24-hour period to an infusion of up to 500 mL or more.

It is still another object of the infusion unit to be used with medication dosing that can be remotely monitored, remote-controlled, and/or with medication easily changed and/or replaced. The infusion unit has a wireless connection to a network so in addition to the ability to adjust medication the vital signs of the patient, namely heart rate, O2 level, blood pressure, and/or glucose levels can be monitored and recorded for review at the location of the patient or at a distal location. Unusual consumption of medication can be detected. Bluetooth and/or Long Term Evolution (LTE) communication can include a local alarm system as well as remote alarm system in case of any error. The communication can include security and protocol to prevent and provide notification of tampering. A global position sensor (GPS) can provide a user, doctor or hospital with a location, telemetry or other information.

In some aspects, the techniques described herein relate to an infusion unit including: a foldable housing configured to be secured around an arm of a person, at least one internal reservoir for temporarily holding a first medication in a bladder; a connection from the at least one internal reservoir to an intravenous (IV) line; a dispensing mechanism that forces a medication from within the at least one internal reservoir through the intravenous (IV) line, wherein the at least one internal reservoir is an IV fluid solution bag that has at least one exit port that connects to the intravenous (IV) line; and a controller that operates the dispensing mechanism based on a predefined parameter.

In some aspects, the techniques described herein relate to a wearable infusion unit including: at least one internal reservoir for temporarily holding a medication in a bladder; a connection from the at least one internal reservoir to an intravenous (IV) line; a dispensing mechanism that forces the medication from within the at least one internal reservoir through the intravenous (IV) line; a controller; and one or more physiological sensors communicatively coupled to the controller and configured to measure one or more vital statistics of a user wearing the wearable infusion unit, wherein the controller is configured to receive the one or more measured vital statistics from the one or more physiological sensors.

In some aspects, the techniques described herein relate to a method of infusing medication into a patient, the method including: securing an infusion unit onto a forearm of a patient; fluidly connecting an intravenous (IV) line to a reservoir connected to one or more drive mechanisms of the infusion unit, wherein the one or more drive mechanisms are configured to force medication from the reservoir and into the IV line; receiving one or more control parameters at a controller communicatively coupled to the infusion unit; and adjusting, according to the one or more received parameters, the one or more drive mechanisms of the infusion unit to adjust a dispensing of the medication from the reservoir of the infusion unit.

Various objects, features, aspects, and advantages of the present invention will become more apparent from the following detailed description of preferred embodiments of the invention, along with the accompanying drawings in which like numerals represent like components.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1A illustrates a schematic representation of an infusion unit.

FIG. 1B illustrates an embodiment of a large infusion unit.

FIG. 2 illustrates a reservoir of an infusion unit embodiment with the drive system.

FIG. 3 illustrates an embodiment of a micro infusion unit.

FIG. 4 illustrates a light case of an infusion unit embodiment with disposable tubing.

FIG. 5 illustrates a drive system of an infusion unit embodiment with a controller.

FIG. 6 illustrates a dispensing barrel of an infusion unit embodiment.

FIG. 7 illustrates a Luer lock syringe of an infusion unit embodiment.

FIG. 8 illustrates a reservoir gear pump of an infusion unit embodiment.

FIG. 9 illustrates a pump of an infusion unit embodiment driving fluid into a vein.

FIG. 10 illustrates a large chamber plunger and chamber of an infusion unit embodiment.

FIG. 11 illustrates a large chamber drive housing for an infusion unit embodiment.

FIG. 12 illustrates a block diagram of the control electronics that may be used for control of an infusion unit embodiment.

FIG. 13 illustrates an electrical block diagram that may be used for control of an infusion unit embodiment.

FIG. 14 illustrates a flow chart for a method of using an embodiment of an infusion unit.

FIG. 15 illustrates a patient wearing an infusion unit embodiment.

FIG. 16 illustrates an embodiment of a pictorial diagram of the doctor interface.

FIG. 17 illustrates a perspective cross-section of an embodiment of the infusion unit.

FIG. 18 illustrates a perspective top view of the flow sensing and control components of an infusion unit embodiment.

FIG. 19 illustrates an embodiment of an infusion unit with a stabilizer.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated embodiments. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention but is merely representative of various embodiments of the invention. The illustrated embodiments of the invention will be best understood by reference to the drawings, wherein like parts are designated by like numerals throughout.

Item Numbers and Description

| | |
|---|---|
| 18 Micro infusion unit | 19 Large infusion unit |
| 20 reservoir | 21 IV-line |
| 22 window | 23 bottom housing |
| 24 plunger | 25 inner layer |
| 26 exit ports | 27 platen swing arm |
| 28 drive bearing(s) | 29 tube platen |
| 30 outer shell | 31 holes |
| 32 outer shell | 33 middle shell |
| 34 plunger cavity | 35 drive cavity |
| 36 motor cavity | 37 bladder cavity |
| 38 bladder/IV bag | 39 circular platter |
| 40 large volume syringe | 41 curved seal |
| 42 plastic tubing | 43 disposable tubing |
| 44 seal | 45 vials |
| 46 plunger(s) | 47 plunger top |
| 50 stepper motor | 51 screw of stepper motor |
| 52 reservoir gear pump | 53 wiring |
| 54 circuit board | 55 display |
| 56 pull tab | 57 anchor tab |
| 60 battery | 62 charging pads |
| 63 transformer | 64 plug |
| 65 touch display | 66 LED(s) |
| 70 controller module | 71 sensor(s) |
| 72 buttons | 73 transmitter/receiver |
| 74 antenna | 75 signal |
| 77 transmitter/receiver | 78 computer |
| 79 display | 80 computer |
| 81 distance | 82, 83, 84 infusion unit |
| 85 hinge | 86 monitor strap |
| 87 lining | 88 security lock |
| 89 patient | 90 main PCB |
| 91 voltage regulator(s) | 92 voltage out |
| 93 connector | 94 air bubble detector |
| 95 flow rate sensor | 96 O2 & pulse sensor |
| 97 ECG sensor | 110 start |
| 111 power on | 112 reset to defaults |
| 113 put syringe | 114 verify syringe |
| 115 display selection | 116 display message |
| 117 wait | 118 verify syringe |
| 119 check selection | 120 route plan |
| 121 default settings | 122 custom settings |
| 123 select rate and type | 124 alert/alarm |
| 125 check field | 127 close lid |
| 128 check the lid | 129 start the process |
| 130 emergency stop | 131 bubble detected |
| 132 stop process | 133 normal process |
| 134 alert message | 135 hold hardware |
| 136 alert/alarm | 137 side connector |
| 140 stop | 145 fluid flow direction |
| 146 rotation direction | 148 female thread mechanism |
| 150 male thread mechanism | 152 aperture |
| 154 circular platter pump | 155 display |
| 156 IV-line access | 157 longitudinal axis |
| 158 controller | 159 angular spacing |
| 160 controller | 162 one or more sensors |
| 164 dispensing mechanism | 166 primary reservoir |
| 168 auxiliary reservoir | 170 auxiliary dispensing mechanism |
| 172 intravenous (IV) line | 174 remote computing device |
| 200 stabilizer | 202 thumb aperture |
| 204 thumb | 100 infusion system |

Embodiments contemplated and described herein are intended for the precise and safe infusion of medical fluids into a patient. Embodiments described herein provide portable and secure devices for the measured and reliable infusion of fluid medication into a patient without the need for hanging poles, external fluid medication bags, unsupported and unprotected tubing, or designated locations of treatment. The infusion units described herein solve the above described problems by including an internal power supply, communications system, and medication distribution syringes or peristaltic pump systems. The infusion units described herein may be intended to be wearable by the patient medication is being infused into.

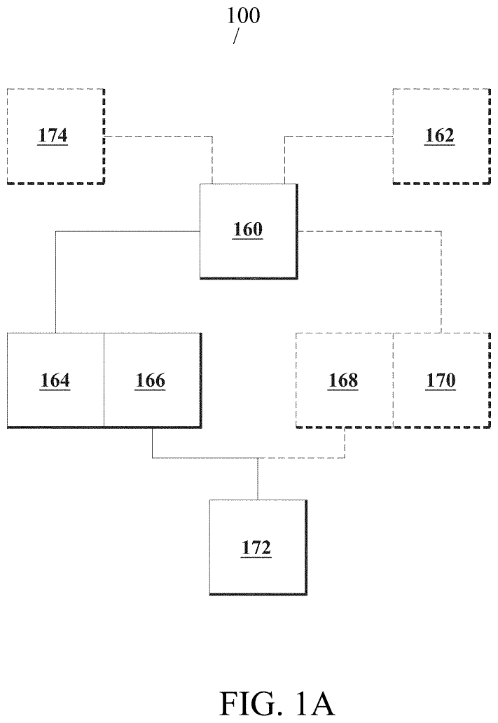

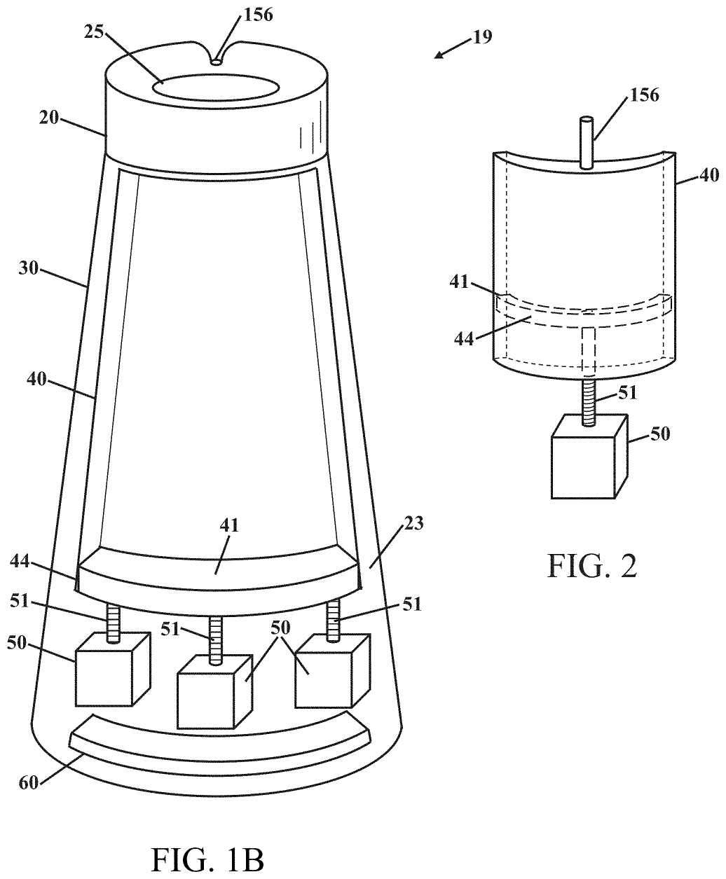

FIG. 1A illustrates a schematic of an infusion system 100. The infusion system 100 includes a controller 160, a primary dispensing mechanism 164, a primary reservoir 166, and an intravenous (IV) line 172. Optionally, the infusion system 100 may include one or more sensors 162, an auxiliary dispensing mechanism 170, an auxiliary reservoir 168, and a remote computing device 174. Although illustrated and described with a primary dispensing mechanism 164 and an auxiliary dispensing mechanism 170, embodiments with more than two dispensing mechanisms and respective reservoirs for temporarily holding medication have been contemplated (e.g., 5 sets, 10 sets, etc.). The controller 160 (may be interchangeable with controller 158 and controller 70) may include processors described herein and may control the actuation of dispensing mechanisms, including, the primary dispensing mechanism 164 and the auxiliary dispensing mechanism 170. The controller 160 may drive fluid from reservoirs (e.g., the primary reservoir 166 and/or auxiliary reservoir 168) at predetermined rates to the intravenous (IV) line 172 to which the reservoirs are fluidly connected. The controller 160 may control the actuation of dispensing mechanisms based on pre-determined parameters, for example, stored in the programmable logic of a processor.

The dispensing mechanisms 170, 164 may be lead screw coupled syringes driven by electric motors (e.g., stepper motors). Alternatively, the dispensing mechanisms 170, 164 may be peristaltic type pumps driven by electric motors (e.g., stepper motors). In a further alternative, the dispensing mechanisms 164, 170 may be biased elements (e.g., a spring coupled to a plunger, an elastic bladder, etc.) that compress the fluid within the primary reservoir 166 and the auxiliary reservoir 168, respectively. Compressing the fluid of a reservoir of the infusion system 100 may eliminate sloshing of the fluid contained by the reservoir. In addition, the controller 160 may selectively actuate valves on the outlet of any compressed reservoir to allow fluid to discharge to the intravenous (IV) line 172. The infusion system 100 may optionally include one or more flow sensors 162, which may actively measure the volume of fluid being discharged from the primary reservoir 166, auxiliary reservoir 168, or any other reservoir into the intravenous (IV) line 172. Alternatively, the infusion system 100 may estimate the volume of fluid discharged from the primary reservoir 166, the auxiliary reservoir 168, or any other reservoir. For example, an embodiment with a lead screw coupled syringe may measure the position of the lead screw with a position sensor 162 or may estimate the position, for example, by tabulating the steps made by a stepper motor driving the lead screw. Another example, an embodiment employing a peristaltic pump may measure (e.g., with position sensors, tachometer, etc.) the amount of pump rotations per time interval (e.g., rotations per minute) and estimate the volume discharged based on a known discharge volume per pump rotation. Embodiments may estimate discharge volumes with volume per rotation and/or may actively measure the discharge volume with one or more flow sensors 162.

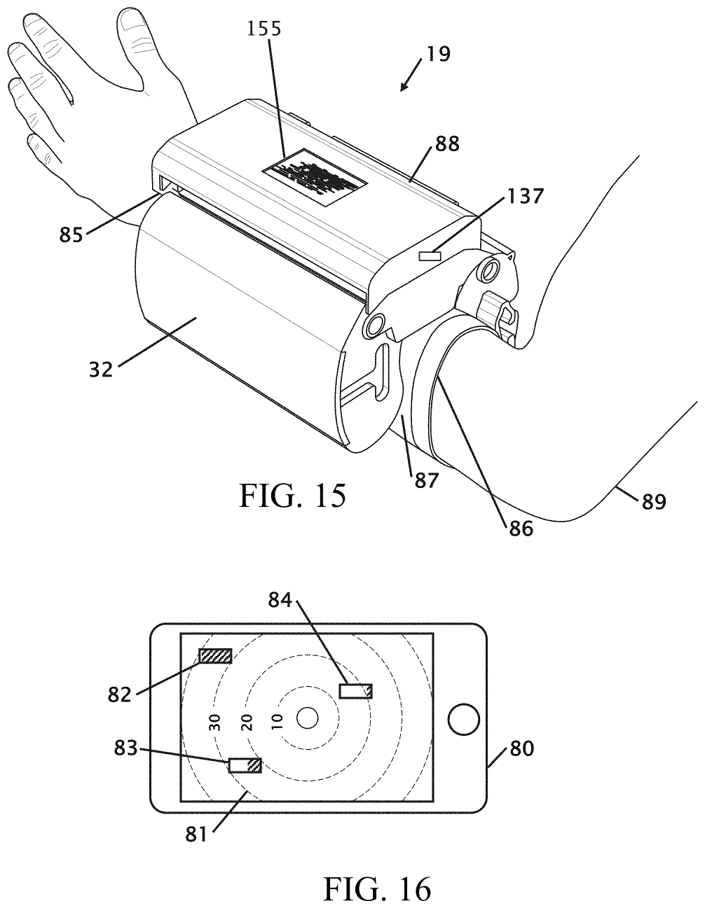

The controller 160 of the infusion system 100 may optionally communicate with a remote computing device 174. Communication between the controller 160 and the computing device 174 may be performed with cellular, satellite, Bluetooth®, Wi-Fi®, Zigbee, or any other appropriate communication networks known in the art. For example, the controller 160 may use Zigbee technology to enable machine-ot-machine and/or internet of thing (IoT) networks. The remote computing device 174 may be a mobile computing device, a server, a handheld remote/controller, a laptop, a VR headset, an unconventionally connected or non-connected device that receives data via email or airdrop, Bluetooth®, etc. Instructions (e.g., control parameters) for dosing requirements or parameters may be input in the remote computing device 174 by a healthcare provider. The remote computing device 174 may transmit instructions that are stored and read by the controller 160. For example, the remote computing device 174 may transmit dosing instructions to the controller 160, which in turn adjust the flow rates of one or more reservoirs. In alternative embodiments instructions (e.g., control parameters) for dosing requirements may be input directly into the controller 160 at a control interface (e.g., the display illustrated in FIG. 15) by a healthcare provider. The controller 160 may transmit data to the remote computing device 174, for example, measured vital statistics from a user of the infusion unit may be transmitted to the remote computing device 174. Optionally, one or more sensors 162 that may be included with infusion units herein may include pressure sensors (e.g., for blood pressure), oxygen sensors, (e.g., O2 sensors), temperature sensors, pulse oximetry sensors (e.g., pulse rate sensor), electrocardiogram sensors, or any other physiological sensors known in the art. As such, the vital statistics of a user wearing an infusion unit may be transmitted to another location, for example, a device monitored by a healthcare provider. In addition to the vital signs of the user, the controller 160 may transmit the fluid data, for example, flow rates from one or more reservoirs, total discharged volumes, fluid volumes remaining in one or more reservoirs, or any other data relating to the present or past performance of the infusion unit. Any or all data transmitted from the controller 160 of the infusion unit may be recorded for the user (e.g., in a user profile).

Monitoring and/or measuring of performance data of infusion units described herein (e.g., flow rates from one or more reservoirs, total discharged volumes, fluid volumes remaining in one or more reservoirs, etc.) may be done in avoidance of undesired dosing (e.g., underdosing or overdosing), or the identification of medication leakage. Additionally, the infusion system 100 may optionally include sensors 162 for the protection of a user of the infusion unit, for example, bubble detecting sensors on the lines between reservoirs and the intravenous (IV) line 172, temperature sensors on mechanisms of the infusion unit (e.g., on electric motors to measure temperatures indicative of failure or electrical short), or any other sensors which measures conditions which may require the shutdown of the infusion unit. The controller 160 may include stored instructions which cause the controller 160 to autonomously shutdown, for example, if a bubble is detected or if the temperature of an infusion unit mechanism is greater than a predefined threshold.

Infusion unit embodiments with two or more reservoirs (e.g., reservoir 166 and 168) may include constant flow capabilities. A concern with infusion devices is the coagulation and/or plugging of the intravenous (IV) line 172 (including the canula). Coagulation and/or plugging is often the result of static conditions within the intravenous (IV) line 172. As such, constant fluid flow through the intravenous (IV) line 172 is effective in reducing coagulation and/or plugging of the intravenous (IV) line 172. For example, if the auxiliary reservoir 168 were to contain maintenance fluid (e.g., consisting of water, glucose, sodium, and potassium), the controller 160 could actuate the auxiliary dispensing mechanism 170 to constantly discharge maintenance fluid through the intravenous (IV) line 172. If the primary reservoir 166 contains a medication to be administered intermittently, constant injection of maintenance fluid from the auxiliary reservoir 168 ensures effective discharge from the primary reservoir 166.

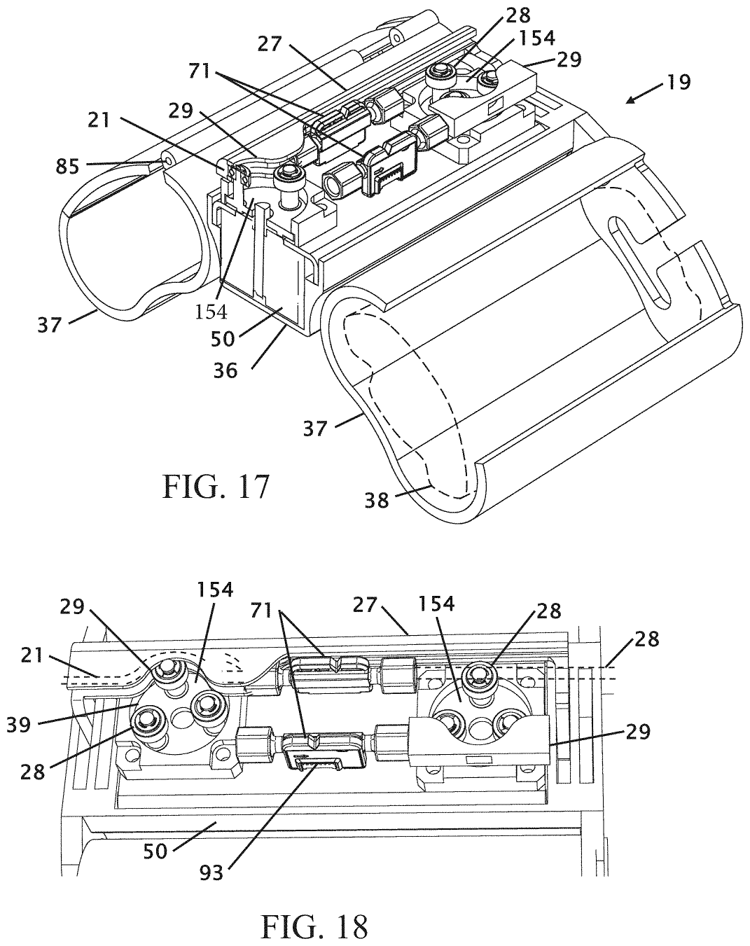

FIG. 1B illustrates a first embodiment of an infusion unit 19. The infusion unit 19 includes an outer shell 30, a large-volume syringe 40, one or more stepper motors 50, one or more lead screws 51, a piston 41, a bottom housing 23, one or more batteries 60, an inner lining 25, and an intravenous (IV)-line access 156. The IV-line access 156 may fluidly connect to the IV-line. The infusion unit has an appearance of an arm cast and may be secured around an arm of a user. Secured around the arm, the infusion unit does not include exposed IV-lines that can be a source of infection, the IV-lines may not be accidentally pulled out of an arm, and infiltration of the IV-lines may not occur. The inner layer 25 includes an interior padding, for example including a soft sponge, Styrofoam lining, gel padding (e.g., polyurethane), or the like, to give the skin maximum comfort, and to keep the infusion unit in place on an arm. Additionally, the infusion unit 19 of FIG. 1B may be coupled with a stabilizer 200 to stabilize the infusion unit on an arm (illustrated and described with respect to FIG. 19). The portion of the infusion unit 19 between the inner layer 25 and the outer shell 30 may include medication compartments with refillable chambers. Large chambers may be in the form of one or more curved syringe compartments for medication. Small chambers may be in the form of one or more micro-syringes. Embodiments may include smaller chambers in quantities of up to 5 or more micro syringes with volumes in a range of about 2 mL to 15 mL; about 3 mL to about 12 mL; about 5 mL to about 10 mL; etc. The outer shell 30 is a cover for protection of the syringes. In an embodiment, the infusion unit is constructed from light-weight materials, for example, a fiberglass material frame with plastic syringes. The infusion unit may be constructed with side hinges (shown and described in FIGS. 15 and 17) allowing a forearm to be placed and secured between two or more portions of the infusion unit.

Illustrated in FIG. 1B, at the top of the infusion unit 19 is a reservoir 20. The reservoir 20 may be made from aluminum or another durable light-weight material. The reservoir 20 is in fluid communication to an IV-line access 156 and the large-volume syringe 40. The IV-line access 156 is recessed within the reservoir 20 where it is protected from an unauthorized person attempting to gain access to the IV-line. Additionally, recessing the IV-line access 21 into the reservoir 20 also keeps the IV-line in an area that prevents accidental damage. The central part of the large infusion unit 19 is an outer shell 30 that includes a cover for protection of the one or more syringes. The outer shell 30 has hinges 85 (shown in FIGS. 15 and 17) and/or locks to encase the forearm and secure the forearm therein. The entire unit is constructed of strong light-weight material (e.g., aluminum, fiberglass, polymers, etc.). Strong light-weight material (e.g., polypropylene, polyethylene, etc.) provides structure for the housing and protection to the internal medication reservoirs.

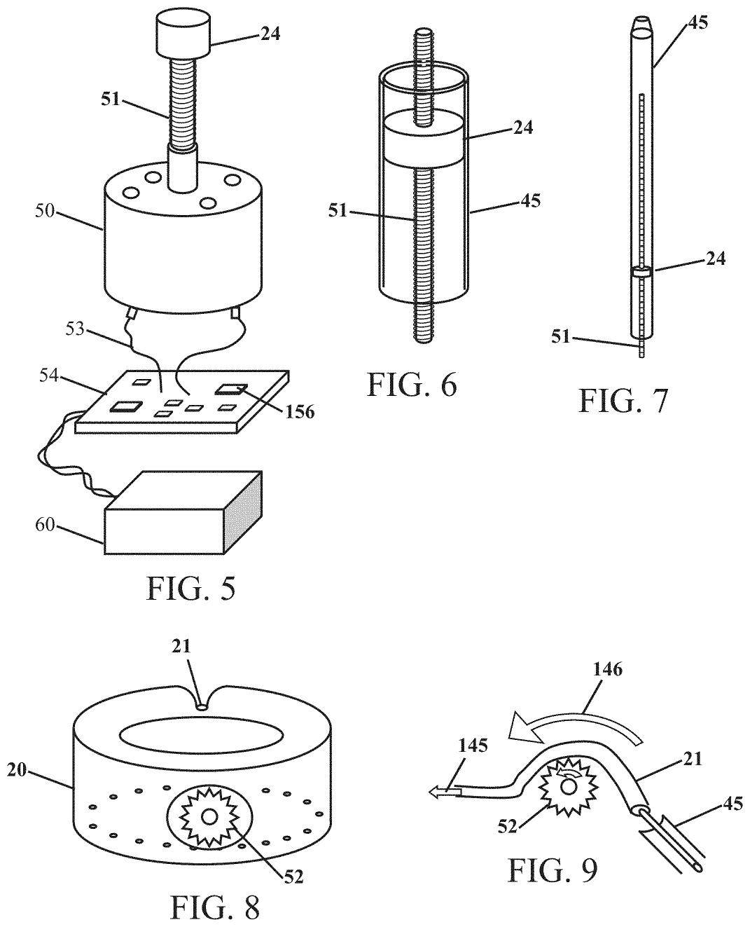

In the embodiment shown in FIG. 1B, the medication is contained within a large volume syringe 40 that has a volume in a range of about 50 mL to about 600 mL; about 100 mL to about 500 mL; etc. The large volume syringe 40 has a defined volume that is curved and 12 includes a curved seal 44 of the piston 41 matching the defined volume. The large volume syringe 40 may be constructed with plastic or any other appropriate material for syringes known in the art. The curved shape of the curved seal 44 of the piston 41 and the curved shape of the large-volume syringe 40 allows the shape of the infusion unit to couple to a patient's forearm while remaining ergonomic and without becoming bulky on the patient. The bottom housing 23 includes one or more stepper motors 50, a controller 158 (shown in FIG. 5), and power supply (e.g., one or more batteries 60). The piston 41 is driven by one or more lead screws 51 from one or more respective stepper motors 50. FIG. 1B shows three stepper motors 50, but as few as one or two may be used to drive the piston 41. Additionally, the one or more lead screws 51 may extend through the piston 41 and into the large-volume syringe 40. The threaded interface between the piston 41 and the one or more lead screws 51 may be sealed as to hold pressure in the large-volume syringe 40 while the piston 41 is pressed into it.

Another version of the infusion unit 19 shown in FIG. 1B may include one or more separate curved chambers located on opposite sides of the infusion unit 19. An embodiment with two curved syringes may include four stepper motors 50, in which two stepper motors 50 are included with each of the two curved syringes. The stepper motors 50 may be coupled to pancake unipolar leadscrew motors. The stepper motors 50 may be powered by a power source, for example a rechargeable battery such as a 12v Lithium battery. The infusion device may reverse drive each stepper motor 50 to reset the syringe to a starting position or a zero position. Using multiple stepper motors 50 on a piston 41 ensures that all portions of the piston 41 are moved uniformly.

FIG. 2 shows a reservoir with a drive system. The drive system includes a stepper motor 50 to precisely advance and retract the piston 41 into and out of the large-volume syringe 40. The drive system also illustrates the IV-line access 156 in which fluid may be pressured out of the large-volume syringe 40 by the piston 41.

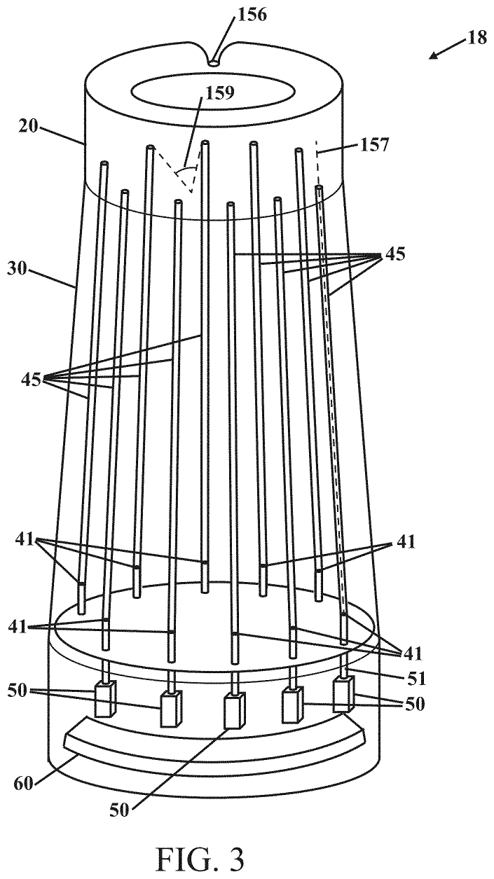

FIG. 3 shows a second embodiment of a micro infusion unit 18. The micro infusion unit 18 may include a plurality of vials 45, a plurality of stepper motors 50, a plurality of lead screws 51, one or more batteries 60, an outer shell 30, a reservoir 20, a plurality of pistons 41, and an IV-line access 156. As a non-limiting example, there can be ten vials 45. The plurality of vials 45 may include an internal volume ranging from about 5 mL to about 50 mL; from about 10 mL to about 40 mL; from about 20 mL to about 30 mL; etc. For example, if the plurality of vials 45 includes a 25 mL internal volume each, then a total volume of 250 mL would be contained in the micro infusion unit 18. Other vial 45 quantities and vial 45 volumes are contemplated and are dependent upon the mechanical design and the amount of medication that is required. Embodiments including micro vials 45 may include equal angular spacing 159 of the micro vials 45 with respect to the circumference of the outer shell 30. When fluid is discharged from the vials 45, the reservoir 20 collects the fluid from the vials 45 and acts as a manifold for the vials 45 into the IV-line access 156. While a single reservoir 20 is shown, there could be multiple reservoirs that fluidly connect the vials 45 to the IV-line access 156. As an example, different medications contained in individual vials 45 may be dispensed throughout the day. In addition, different mixtures of medications may be dispensed and mixtures may be autonomously adjusted, for example, in the evenings a mixture suitable to assist a person with sleep may be infused into the patient.

As shown in FIG. 3, a plurality of stepper motors 50 are used in the embodiment. In a non-limiting example, the embodiment shown uses ten stepper motors 50 located at the bottom of each vial 45, with five stepper motors 50 placed on each side of the infusion unit 18. Each stepper motor 50 is operatively coupled to a lead screw 51. Each lead screw 51 is threaded into a syringe piston 41, and, as such, the syringe piston 41 is advanced when the lead screw 51 is rotated in a first direction about its longitudinal axis 157. The syringe piston is retracted when the lead screw 51 is rotated in a second direction about its longitudinal axis 157. The threaded connection of the lead screw 51 and the syringe piston may be liquid tight to effectively pressure fluid and dispense the fluid from the vial 45 volume into the reservoir 20. Once the syringe piston has been advanced to its fully advanced position and the medication has been depleted from the volume, the lead screw 51, rotated in the second direction by the stepper motor 50, may be retracted and returned to the initial position. The stepper motors 50 may be communicatively coupled to one or more batteries 60. The term “communicatively coupled” may be defined as either wireless communication (i.e., wirelessly coupled) between components or a wired connection between components. Control elements, for example, one or more H-bridges may be used to control the power levels and polarity supplied from the one or more batteries 60 to the stepper motors 50. While five vials 45 on each side of the injection unit 18 are shown, embodiments with more than five vials 45 per side or less than five vials 45 per side are also contemplated.

The embodiment shown in FIG. 3 may include syringes with internal volumes of about 2 mL to about 3 mL to give a total volume of between about 20 mL and about 30 mL, but syringes with larger or smaller internal volumes can also be used. The stepper motor 50 pushes the syringe pistons one at a time or in any sequence. As such, any combination or mixture ratio may be achieved at the IV-line access 156. Some embodiments may include sensors or methods that detect or recognize when syringes are empty or if air is present in the syringe and/or the delivery line. For example, stepper motors are known in the art to actuate in highly precise and repeatable steps. The steps may be measured in degrees of rotation about the output shaft of the stepper motor. As such, a predetermined amount of steps, for example, may be assigned for full discharge of a syringe. Presence of air may be detected acoustically. For example, two ultrasonic transducers that work as a transmitter and a receiver, respectively, may be placed on opposing sides of tubing and/or a syringe. The two ultrasonic transducers may measure the acoustic impedance of the fluid between the two transducers. When the acoustic impedance measurements are received by a controller 158 (shown in FIG. 5), the acoustic measurements are compared to predefined values for medication fluids and air to determine if air is present in the tubing and/or syringe.

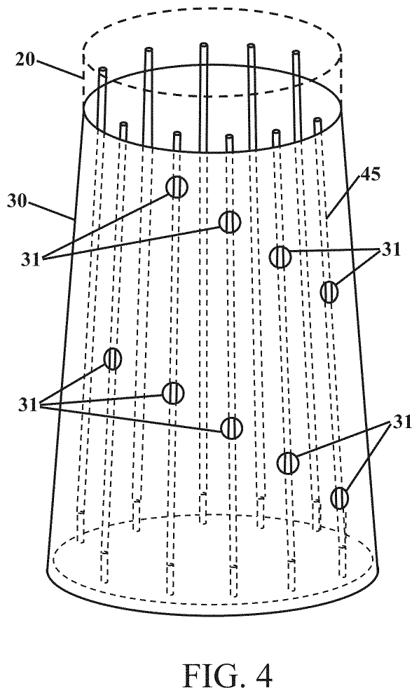

FIG. 4 illustrates the embodiment of FIG. 3 including a light-weight outer shell 30, vials 45 constructed of disposable tubing, and a reservoir 20 shown in broken lines at the dispensing end of the vials 45. The outer shell 30 includes a plurality of holes 31 that each provide a view of a portion of a vial 45. The outer shell 30 may be constructed of a material that provides structural strength while being light weight, for example, aluminum. Aluminum is not transparent, as such, the plurality of holes 31 may be placed through the outer shell 30 to allow viewing of the level of fluid within the vials 45 without opening the infusion unit. Alternatively, a durable and light weight transparent material may be used. The holes 31 are shown in a spiral pattern but could also be placed linearly along the vials 45. Additionally, the outer shell 30 may also include indicia near the holes 31 to allow for a visual measurement of the remaining medication in the vials 45. For example, an indicia of “50%” may be placed on the outer shell 30 near a hole 31. If fluid in a vial 45 is seen only below the hole 31 with the “50%” indicia, it may be assumed that less than half the total fluid remains in the associated vial 45.

FIG. 5 illustrates a drive system of an infusion unit. The drive system includes one or more batteries 60, a circuit board 54, electrical wiring 53, a stepper motor 50, a lead screw 51, and a plunger 24. The circuit board 54 may include a controller 158 that receives power from the one or more batteries 60 communicatively coupled to the circuit board 54. The controller 158 may include a processor capable of receiving multiple-inputs and outputting multiple outputs. For example, a Arduino micro-processor, Nordic® micro-processor, or any other micro-processor may be used to receive multiple inputs and to generate multiple outputs. Additionally, the circuit board 54 may include control elements, such as relays, H-bridges, or any other electric motor control elements known in the art. The circuit board 54 then couples to the stepper motor 50 via electrical wiring 53. The batteries 60 may be replaceable or may be rechargeable using a charging device or an inductive charger. Stepper motors 50 provide a high level of accuracy. The output shaft of the stepper motors 50 may be coupled to the lead screw 51. The lead screw 51 may be operatively coupled to the plunger 24 at a threaded interface. If the plunger 24 is held from rotating with the lead screw 51, as it is rotated by the stepper motor 50, the lead screw 51 may incrementally advance or retract the plunger 24. The plunger 24 may be restrained from rotating with the lead screw 51 due to the shape of the plunger 24 and the vial 45 shape. For example, an oval plunger within an oval vial 45 is not conducive to rotation with respect to the vial 45.

FIG. 6 illustrates a dispensing barrel vial 45. The dispensing barrel vial 45 includes a lead screw 51 and a plunger 24. FIG. 6 illustrates the dispensing capability of the vial 45 when lead screw 51 is rotated in a first direction, as well as the retraction capability of the plunger 24 when the lead screw 51 is rotated in a second direction, opposite the first direction. The rotation of the lead screw 51 by the stepper motor 50 (shown in FIG. 5) and resulting advancement of the plunger 24 will be ceased when it reaches a predefined limit, for example a fully dispensed position. When the plunger 24 reaches the predefined limit, the controller 158 (shown in FIG. 5) may provide an inverse polarity electrical current (i.e., inverse to the polarity used to advance the plunger 24) to the stepper motor 50, to cause the stepper motor 50 to rotate the lead screw 51 in the opposite direction to retract the plunger 24. When the plunger 24 is caused to retract, it may continue to retract until it reaches a predefined position, for example, an initial position. An initial position, as in the previous example, may be recognized by the controller 158 (shown in FIG. 5) due to the quantity of steps between a plunger 24 at the predefined limit (e.g., fully dispensed position) versus a plunger 24 at the initial position. For example, if the stepper motor rotates through 480 steps to move the plunger to a fully dispensed position (shown in FIG. 5), the controller (shown in FIG. 5) would generate 480 steps in the opposite direction to return the plunger 24 to the initial position (e.g., once disconnected from the IV-line).

FIG. 7 shows a Luer lock syringe. The Luer lock syringe may include a lead screw 51, a vial 45 and a plunger 24. The advancement and retraction of the plunger 24 may be performed as described for FIG. 6. Advancing the plunger 24 dispenses fluid from the vial 45 and retracting the plunger 24 may return the plunger 24 to an initial position.

60 mL syringes (or other volume) may be placed in an infusion unit embodiment which fits on an adult forearm with a tube connected to an IV-line. The infusion unit supports and contains one or more stepper motors 50 and a controlling module. The stepper motor 50 has lead screw 51 attached to the piston 24 of the syringe (shown in FIG. 3). This embodiment may be used with low volume precision infusions of antibiotics, hormonal replacements, and other low volume drugs. The range of infusion of some embodiments described herein may be about 0.5 mL/hr to about 150 mL/hr; about 1 mL/hr to about 100 mL/hr; about 2 mL/hr to about 80 mL/hr; etc. The described rates of infusion are commonly used for patients who need antibiotics at home or in a nursing home or at a hospital.

FIG. 8 shows a gear pump 52 of a reservoir 20. Any embodiments described herein may include one or more gear pumps 52 within the reservoir 20. One or more vials 45 (as shown in FIGS. 3 and 4) may be fluidly coupled to one or more gear pumps 52 within the reservoir 20. Some embodiments include a gear pump 52 for each vial 45. When fluid is dispensed from a vial 45, the gear pump 52 may be activated to increase the pressure of the fluid for injection into a vessel. The fluid expelled from a respective vial 45 may be pressurized and injected into the IV-line 21 by the gear pump 52. Alternatively, the gear pump 52 may be used to create suction in respective vial 45 and may pump incoming fluid from the vial 45 into the IV-line 21. Multiple lines coming from multiple gear pumps 52 may manifold into the IV-line 21. FIG. 9 illustrates the gear pump 52 driving fluid into the vessel of a patient. FIG. 9 illustrates the direction of rotation 146 of gear pump 52 to accomplish the indicated fluid flow direction 145. The gear pump 52 of FIGS. 8 and 9 may perform substantially the same as gear pumps known in the art.

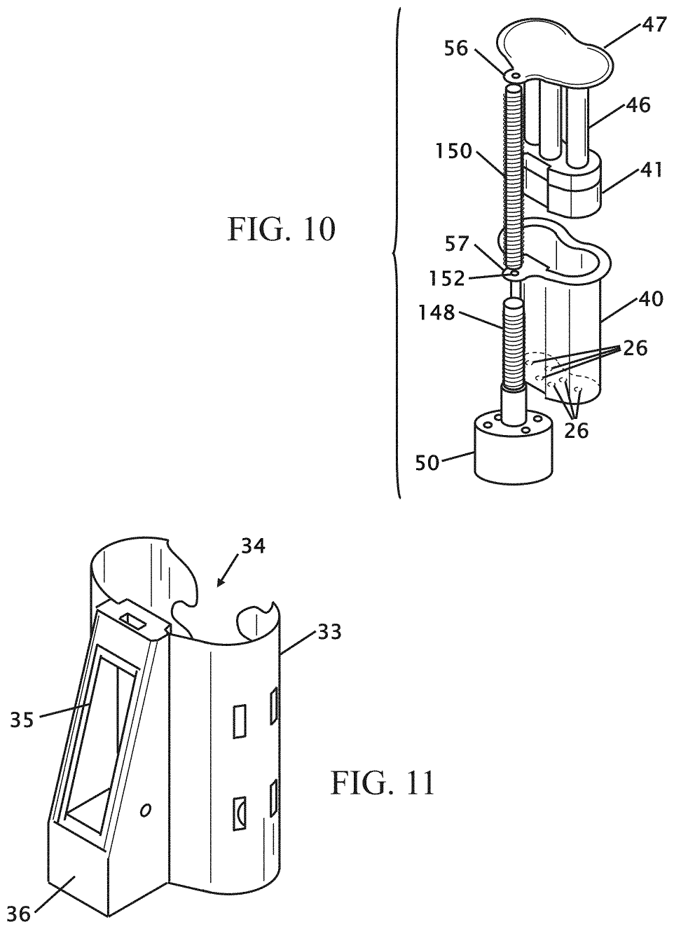

FIG. 10 illustrates a high-volume pump (FRN) including a syringe 40 with an anchor tab 57, a curved seal 41, a plunger 46, a plunger top 47 with a pull tab 56, a lead screw 51, and a stepper motor 50. The syringe 40 includes one or more exit ports 26 opposite the end that the curved seal 41 enters the syringe 40. The embodiment of FIG. 10 may also be referred to as a high-volume pump (FRN). The embodiment of FIG. 10 uses a kidney shaped syringe 40 (MRZ) with a capacity in a range of about 100 mL to about 700 mL; about 200 mL to about 600 mL; about 300 mL to about 500 mL; etc. The kidney shape of the syringe 40 is well-suited for placement of the syringe 40 on a forearm of a patient. A kidney shape is defined herein as two bulbs or bulbous sections joined together by a narrowed intermediate portion. The narrowed intermediate portion and two bulbous sections create a concave portion found on one side of the shape but not found on the opposite side of the shape. The stepper motor 50 rotates a female thread mechanism 148 that engages the male thread mechanism 150. The male thread mechanism 150 is coupled to the pull tab 56. The anchor tab 57 of the syringe 40 may define an aperture 152. The diameter of aperture 152 may be greater than the diameter of the male thread mechanism 150 and may be less than the diameter of the female thread mechanism 148, thus, the male thread mechanism 150 may translate through the aperture 152. Rotating the female thread mechanism 148 in a first direction pulls the male thread mechanism 150 into the female thread mechanism 148. The male thread mechanism 150 may pull the pull tab 56 of the plunger top 47, which pulls the plunger 46 and curved seal 41 into the syringe, thus, pushing fluid out of the syringe into the tubing. The tubing that fluid is pushed into may be plumbed into the IV-line and, ultimately, into a vessel of a patient. The bottom of the large volume syringe 40 may include a plurality of exit ports 26 to decrease the thrust pressure and increase the desired volumetric flow rate output. The multiple exit ports 26 may be combined into a single IV-line. An example use case for this embodiment may in the field of EMS for injured patients or in a battlefield for soldiers.

FIG. 11 illustrates a middle shell 33. The middle shell 33 may include a plunger cavity 34, a motor cavity 36, and a drive cavity 35. The middle shell 33 may be dimensioned and intended to house the high-volume pump (FRN) described in FIG. 10. The plunger 46 and syringe 40 may fit within the plunger cavity 34. The stepper motor 50 may fit within the motor cavity 36. The male thread mechanism 150 and female thread mechanism 148 connected, respectively, to the tabs 56, 57 on the plunger and syringe, may fit within the drive cavity 35.

The high-volume infusion unit, illustrated in FIG. 10, may include the high-volume pump (FRN) described in FIG. 10 housed within the middle shell described in FIG. 11. The high-volume infusion unit may include a prefilled syringe with an internal volume within a range of about 250 mL to about 750 mL; about 300 mL to about 600 mL; about 350 mL to about 450 mL; etc. The high-volume infusion unit may be positioned on a portion of a patient's forearm, for example, on the dorsal aspect of the forearm. A stabilizer (e.g., plastic board) on the other side of the forearm may be used to secure the high-volume infusion unit by hook and loop bands on both sides of the stabilizer. Additionally, or alternatively, the high-volume infusion unit may utilize the stabilizer 200 (illustrated in FIG. 19) to stabilize the infusion unit on the forearm. The ease of placement and securement of the high-volume infusion unit may optimize it for use in the field, such as, a battlefield with injured soldiers. By using the forearm as the securement structure and eliminating the need for gravitational forces to flow fluids into a patient, the need for a pole and IV bag is eliminated.

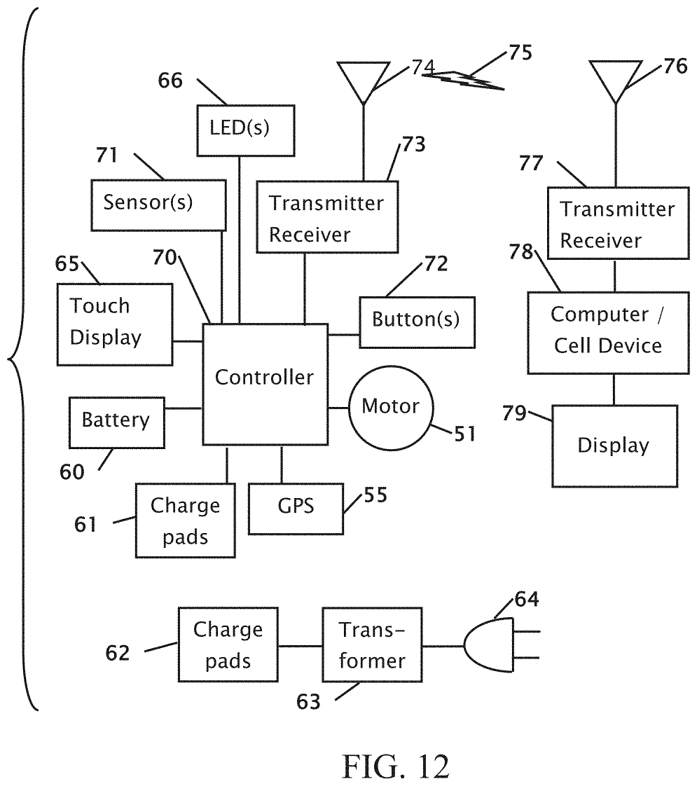

FIG. 12 illustrates a block diagram of the control electronics. A first subsystem of the block diagram illustrated in FIG. 12 may include a controller 70, one or more sensors 71, one or more light indicators 66 (e.g., LEDs), a transmitting and receiving device 73, an antenna 74, one or more input mechanisms 72 (e.g., button), one or more stepper motors 51, an optional global positioning sensor 55, a charging pad 61, one or more batteries 60, and an optional display 65. A second subsystem (e.g., an external communication device) of the block diagram illustrated in FIG. 12 may include a display 79, a remote computing device 78, a transmitting and receiving device 77, and an antenna 76. A third subsystem of the block diagram illustrated in FIG. 12 may include one or more charge pads 62, a transformer 63, and an electrical plug 64.

The first subsystem of the block diagram illustrated in FIG. 12 may be representative of any infusion unit embodiment described herein. The controller 70 may include a processor based control system (e.g., a microcontroller) to control the dispensing of medication. The control system may further include one or more processors that may receive multiple inputs and generate multiple outputs. The controller 70 may include input mechanism(s) 72 and an optional display 65 (e.g., a touch display) that can be used as a user interface with the infusion unit. The input mechanism(s) 72 and optional display 65 (e.g., a touch display) may be used to generate signals that, when received by the controller 70, cause the controller 70 to generate control outputs. For example, the input mechanism(s) 72 and optional display 65 (e.g., a touch display) may be used to generate inputs to the controller 70 that cause the controller 70 to output signals that increase a flow rate of the infusion unit, decrease a flowrate of the infusion unit, cease infusion procedures, and, in embodiments with multiple syringes or reservoirs, change the mixture of medication infused in the patient. Some embodiments may include an optional display 65 that is only for display purposes. The one or more light indicators 66 may be used to indicate statuses of the infusion unit. For example, a status indicated by a light indicator 66 may be whether or not the voltage levels of the power source 60 are above an acceptable level, or if the amount of fluid within a syringe is lower than a predefined threshold. The one or more sensors 71 may monitor fluid levels in one or multiple syringes or reservoirs. Additionally, one or more sensors 71 may monitor a patient's vital signs, including but not limited to, heart rate, blood oxygen saturation, and blood pressure. Additionally, the one or more sensors 71 may include sensors that can detect when a syringe has air in it or when a syringe is empty. Further, the one or more sensors may include bubble detecting sensors for tubing leading to the IV-line. The infusion unit may include an optional global positioning sensor 55 to track the patient and the infusion unit for security reasons and/or to easily locate a patient. The infusion unit may be powered by one or more power sources 60 (e.g., batteries) that may be recharged with charging pads 61 that may enable physical connection to an electrical source or may include inductive charging elements. The infusion unit is self-contained, but in some embodiments may include a transmitting and receiving device 73 with an antenna 74. The transmitting and receiving device 73 may receive signals from the controller 70 and transmit the signals to the remote computing device 78 of the second subsystem. The transmitted signals from the first subsystem may include, for example, the fluid levels in one or multiple syringes or reservoirs, the patient vital signs, and/or the patient's position as measured by the global positioning sensor 55. Additionally, the transmitting and receiving device 73 may receive signals from a remote computing device 78 and transmit them to the controller 70. The signals transmitted from the transmitting and receiving device 73 may be received by the controller 70 as inputs that cause the controller 70 to output control signals, for example, that increase a flow rate of the infusion unit, decrease a flowrate of the infusion unit, cease infusion procedures, and, in embodiments with multiple syringes or reservoirs, change the mixture of medication infused in the patient.

The second subsystem of the block diagram illustrated in FIG. 12 may be used to receive and transmit signals to the first subsystem (i.e., an infusion unit). The second subsystem may include a remote computing device 78 which may receive signals from a user interface and display information from the first subsystem on the optional display 79. For example, a physician may change the mixture of medication to be infused into the patient. The signal may be processed by the remote computing device and transmitted by the transmitting and receiving device 77 to the first subsystem. The first subsystem (e.g., an infusion) may react by changing flowrates of syringes or reservoirs therein. The second subsystem may receive signals from the first subsystem indicating, for example, the fluid levels in one or multiple syringes or reservoirs, the patient vital signs, and/or the patient's position as measured by the global positioning sensor 55. Signals received from the first subsystem by the second subsystem may be processed by the remote computing device 78 and displayed on the display 79. The remote computing device 78 may be a mobile computing device, a server, a handheld remote/controller, a laptop, a VR headset, an unconventionally connected or non-connected device that receives data via email or airdrop, Bluetooth®, etc.

The third subsystem of the block diagram illustrated in FIG. 12 may be used to charge the first subsystem (e.g., an infusion unit). One or more charge pads 62 may be powered by a transformed electrical current from the transformer 63. The transformer 63 may receive an input electrical current from the electrical plug 64, plugged into an electrical circuit. A charging pad 62 physically connected to the charging pad 61 (i.e., plugged into) of the first subsystem, or inductively coupled to the charging pad 61 of the first subsystem, may be used to transfer electrical current to the one or more power sources 60 of the first subsystem.

The second subsystem may be a programming and/or monitoring device. Examples of a remote computing device 78 may include a computer, a cellular device, a wireless device, or a tablet. The communication between the transmitting and receiving device 73 of the first subsystem and the transmitting and receiving device 77 of the second subsystem may be conducted with Bluetooth®, WI-FI®, Zigbee, a cellular connection, or any other communication technology known in the art.

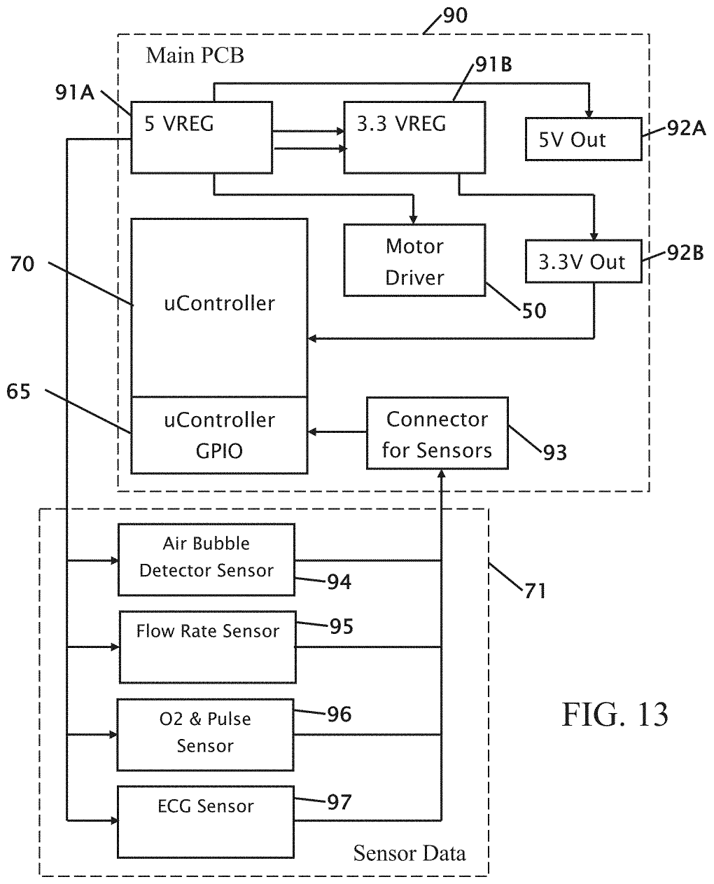

FIG. 13 illustrates an electrical block diagram that may represent the control system of any infusion unit embodiment described herein. The electrical block diagram includes a main printed circuit board (PCB) block 90 and a sensor data block 71. The main PCB block 90 includes a 5-volt regulator 91A, a 3.3-volt regulator 91B, a motor driver 50, a controller module 70, an optional touch display 65, and a connector 93. The sensor data block 71 may include an air bubble detection sensor 94, a flow rate sensor 95, an electrocardiogram sensor 97, and one or more auxiliary sensors 96. The one or more auxiliary sensors 96 may include at least one of the following: an O2 sensor, a pulse sensor, and/or a blood pressure sensor. Unregulated voltage is regulated with a 5-volt regulator 91A, producing a 5-volt output 92A that may be used for powering the motor driver 50. A 3.3-volt regulator 91B regulates the unregulated voltage to a 3.3-volt output 92B that may be used to power the sensors and the controller module 70. The controller module 70 is shown connected to an optional touch display 65 to display information to a doctor, user, or a caregiver and to also allow for the user to select and enter information. A connector 93 provides an electrical connection from the controller module 70 to the sensors.

The sensor data block 71 has connections for an air bubble detection sensor 94 that can detect if air is in the IV-line or the tubing proceeding the IV-line. A flow rate sensor 95 may be used to ensure that medication is being supplied at the prescribed rate. The infusion unit may also monitor vital signs of a patient with the one or more auxiliary sensors 96, which may include at least an O2 sensor, pulse sensor, and/or blood pressure. Additionally, an electrocardiogram sensor 97 may be used to also monitor the vital signs of a patient.

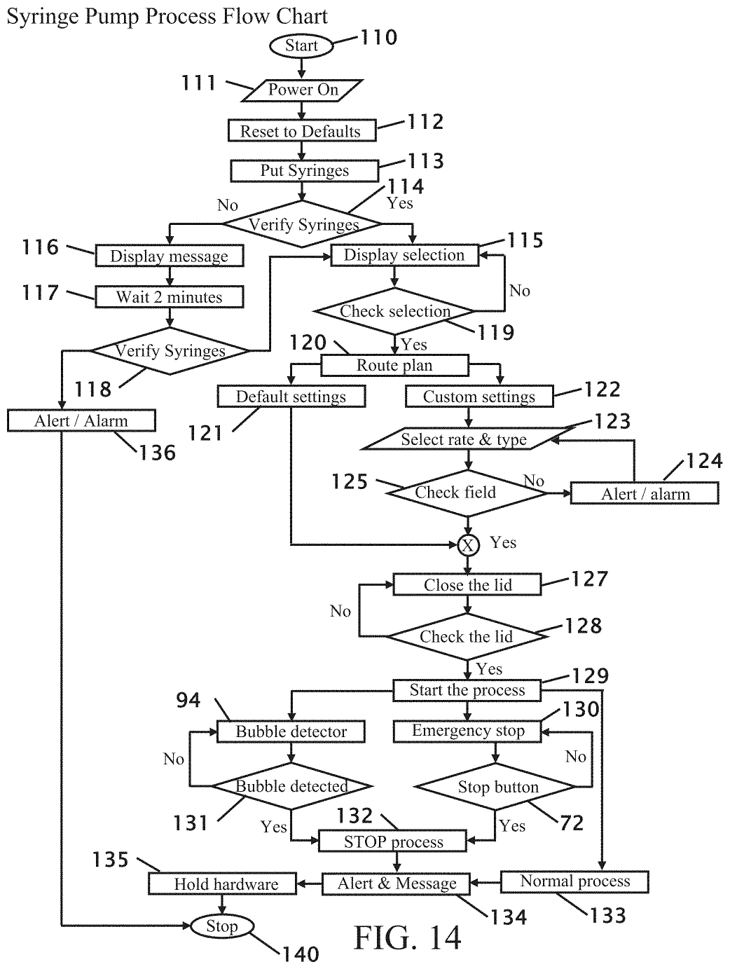

FIG. 14 illustrates a flow chart for a method of using an infusion unit. The method of using the infusion unit starts at block 110 when power is turned on at block 111. As the infusion unit starts, it resets all settings to default settings at block 112. The infusion unit will check if at least one syringe is installed at block 113 and can verify the presence of at least one syringe at block 114. For example, the infusion unit may scan for a Radio-frequency identification (RFID) chip installed on a syringe, and if an RFID chip is recognized, the presence of at least one syringe is verified. If a syringe is not verified, a message will be displayed on the display screen at block 116. The infusion unit will wait for a period of time, in this example two minutes at block 117, and will again check if a syringe has been installed by verifying the presence of at least one syringe at block 118. If a syringe has not been installed, an alert or alarm may be shown or an audible alarm may be generated by one or more speakers at block 136. While a two-minute delay is shown at block 111, any appropriate delay period may be used.

If a syringe is detected, the infusion unit may include a scanner that may read the type of medication from a label, a barcode (or any other optical identifier) or RFID chip indicator on the medication syringe or IV medication bag associated with the contents therein. Scanners used in infusion unit embodiments described herein may be of any variety known in the art. The infusion unit may match the barcode or RFID chip to a suggested medication or to a patient record to load settings. A selected syringe installed in the infusion unit may be displayed at block 115 and can cross-check with the prescribed selection at block 119. The selection will identify a route plan at block 120. The doctor or other care provider may make a custom setting at block 112 or allow for the default setting at block 121. In the custom setting at block 122, the rate and type of the medication is entered at block 123.

One or more rates of injection are checked (e.g., verified by one or more flow sensors), set or selected at block 125, and if all or a required subset of the data is not properly entered, an alert or alarm may be shown or an audible alarm may be generated by one or more speakers at block 124. The infusion unit may verify that the lid of the infusion unit has been closed at block 128. If the lid is not found to be closed, the user may be prompted to close the lid on the display screen at block 127. For example, the lid may include a reed switch and the infusion unit may include a magnet in a position proximal to the reed switch when the lid is closed. If the controller receives a signal across the reed switch (i.e., the reed switch is closed), the lid is registered as closed by the controller. If a signal cannot be carried across the reed switch (i.e., the reed switch is open), the lid is registered as open by the controller. The infusion unit must verify that the lid is closed before allowing the infusion unit to start the process of injecting the medication at block 129.

While the medication is being injected at block 129 the infusion unit will monitor the flow of medication, checking the IV-line for air bubbles at block 94. If an air bubble is detected at block 131, via an air bubble detection sensor, the process is stopped at block 132. The process can also be stopped if an emergency stop button is pressed at block 72, instructing an emergency stop at block 130. One or both of the events at blocks 131 and 72 can stop the process at block 132. There are other contemplated scenarios where the process can be stopped, including, but not limited to, the normal process completing at block 133, if the medication stops flowing, or if the vital signs of the person exceed a prescribed safety level.

When the process of the infusion unit stops, an alert may be generated, and a message may be displayed at block 134. The hardware is placed on a hold position at block 135 to prevent further injection of medication and the process will come to a stop at block 140.

FIG. 15 shows a patient wearing an infusion unit embodiment. The embodiment of FIG. 15 includes an infusion unit 19 as described herein, a lining 87 (e.g., Styrofoam), a monitor strap 86, one or more side hinges 85, a security lock 88, a side connector 137, and an optional display 155. As illustrated, the infusion unit 19 is aesthetically similar to a cast on an arm 89 of a patient. The infusion unit is secured without tubing or the IV-line exposed, thus, lowering the chance of infection or accidental pulling on the tubing or IV-line. The outer shell 32 of the infusion unit may be a cover with protection for the internal syringes. The middle or intermediate layer may include one or more medication compartments with refillable curved syringe compartments or micro-syringes. The interior includes lining 87 (e.g., a soft sponge Styrofoam) to provide skin comfort and keep the infusion unit 19 in place. The infusion unit 19 may remain light-weight due to use of e.g., fiberglass material and plastic syringes, as described herein. The infusion unit may include at least one portion that is coupled to another portion by hinges 85. Thus, making the housing of the infusion unit foldable, and the forearm may be placed between two or three portions of the infusion unit that hinge together, securing the infusion unit to the forearm. A security lock 88 holds the hingedly coupled portions of the infusion unit together and prevents tampering within the infusion unit. Access through the security lock 88 may require a unique manual code entered into the infusion unit by a keypad or through the optional display 155 (e.g., a touch display). Additionally, access through the security lock may be performed with a remote computing device. For example, a verified user on a remote computing device may unlock the security lock 88. The infusion unit may include the one or more sensors 71 described for FIG. 12 that include a pulse sensor, a flow meter, an air bubble detector, and/or a EKG sensor.

Within the infusion unit is a lining 87 to provide skin comfort and keep the infusion unit 19 in place on the forearm. The infusion unit is illustrated with a monitor strap 86 that may include a heart rate sensor, an O2 sensor, and/or other patient vital sign sensors. Vital sign sensors placed at the monitor strap may monitor vital signs with minimal noise and motion interference from the one or more drive motors of the infusion unit. It has also been contemplated that a thumb hole defined by a portion of the infusion unit may be implemented to further lock the infusion unit onto the forearm of the patient and to prevent rotation of the infusion unit. Preventing rotation of the infusion unit may reduce the chance that the IV is disturbed. The infusion unit may have a security lock 88 to prevent tampering or removal. Additionally, infusion units described herein may include tamper detection capabilities. For example, infusion units described herein may include a seal between two or more portions of the infusion unit (e.g., the portions hinged coupled together). Access into the infusion unit may be require the breaking of the seal. The seal may be a sacrificial single use seal; in which the seal is damaged upon opening the infusion unit. Upon inspection, a damaged seal may indicate to a person of authority that unauthorized access has occurred. Further embodiments with active tamper detection have also been contemplated. For example, infusion units described herein may include a sealed interface between two or more portions (e.g., the portions hingedly coupled together) and may include one or more tamper detection sensors within the sealed interface. Tamper sensor types may include humidity, ambient light, hall-effect, or any other appropriate sensor types. Respectively, the sensor types may measure changes in humidity, ambient light, and magnetic field that may be indicative to tampering (e.g., opening the infusion unit case may change humidity, allow in more ambient light, etc.). Upon receiving a tamper sensor measurement greater than a predetermined threshold, the controller may alert a person of authority that un-authorized access has occurred. FIG. 15 illustrates an optional display 155 or touch display and buttons (not visible) that may be used for programming or locally viewing information regarding the medication within the infusion unit 19.

A doctor or other medical care provider may view statuses or make changes using a remote computing device, such as, a computer, a tablet, or, in FIG. 16, a cellular phone 80. FIG. 16 illustrates a display that may be generated upon, for example, a cellular phone 80. The display includes rings indicating the distance 81 between the cellular device 80 and a plurality of infusion units 82, 83 and 84. Each of the infusion units 82, 83, and 84 illustrated may include a graphical image of the remaining medication. A doctor or other medical care provider may use a remote computing device to select an individual infusion unit to view additional information and make changes to the setting of an infusion unit remotely. Changes made from a remote computing device may be logged, time stamped, and stored for future reference. It has also been contemplated that the infusion unit may include a side connector 137 (shown in FIG. 15) to communicatively couple auxiliary sensors to the infusion unit. For example, one or more auxiliary sensors that may be communicatively coupled to the infusion unit may include sensors (e.g., placed on a patient's chest) that measure respiratory rate, blood pressure, blood analysis, glucose lactate information, and/or any other useful vital sign information.

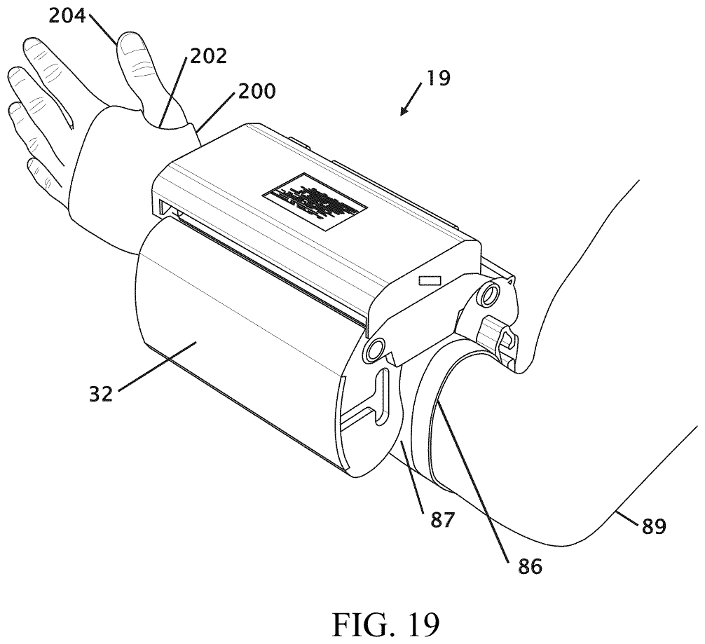

FIG. 17 illustrates a perspective view of another embodiment of an infusion unit. In FIGS. 17 and 18, the top housing, the display, and control components have been removed to view the internal structure and assemblies. The infusion unit of FIGS. 17 and 18 includes an infusion unit 19, one or more bladder cavities 37, one or more sensors 71, one or more circular platter pumps 154 (e.g., peristaltic pumps), one or more platen swing arms 27, a motor housing 36, and one or more tube platens 29. The embodiment of the infusion unit shown in FIG. 17 includes portions which fold on one or more hinges 85 around an arm of a user (e.g., shown in FIG. 15). The bladder cavities 37 are shown in FIG. 17 in an open configuration. The one or more bladder cavities 37 may contain a bag or common IV fluid solution bag 38. A fluid solution bag 38 filled with medication may be placed within one or both of the bladder cavities 37. An IV-line 21 may be connected to the fluid solution bag 38, with the IV-line 21 leading into the delivery system of the motor housing 36. The fluid solution bags in this embodiment are 250 mL or less bags (e.g., 250 mL piggyback bags).

FIG. 18 shows a perspective top view of the flow sensing and flow control components. Within the motor housing 36, there may be one or two dispensing mechanisms and sensing units. In FIGS. 17 and 18, the IV-line 21 passes through a respective sensor of the one or more sensors 71 that monitor the flow rate, presence of air bubbles, and/or any other parameters described herein. A connector 93 on the one or more sensors 71 may connect each sensor into the controller to be represented on a display (control module and display not shown in these figures). The medication from the one or more fluid solution bags 38 is drawn (or pushed) through a respective IV-line 21 and past or through a respective sensor of the one or more sensors 71. Fluid from the one or more fluid solution bags 38 is pumped through a respective IV-line 21 by the one or more circular platter pumps 154. The one or more platter pumps 154 may include a stepper motor 50 that turns one or more drive bearings 28 rotating on a circular platter 39. The IV-line 21 follows and is restrained within the platen swing arm 27. Adjacent to the platen swing arm 27 may be the tube platen 29. The tube platen 29 may include a semi-circular path constraining the IV-line 21 between the tube platen 29 and the rotation path of the one or more drive bearings 28. The spacing between the one or more drive bearings 28 and the tube platen 29 may be less than the diameter of the IV-line 21 when a drive bearing of the one or more drive bearings 28 rotates past the tube platen 29. As such, when the stepper motor 50 rotates the circular platter 39 to which the one or more drive bearings 28 are coupled, a drive bearing of the one or more drive bearings 28 contacts the IV-line 21 as it rotates past the tube platen 29. The contact of the drive bearing 28 squeezes the IV-line 21 to pump fluid from the fluid solution bag 38 (shown in FIG. 17) in the direction of the passing drive bearing 28. The IV-line 21 (e.g., a thermoplastic polymer) expands back to its original shape after the drive bearing of the one or more drive bearings 28 passes, refilling with fluid from the fluid solution bag 38. Refilled with fluid, the IV-line 21 may be recompressed by a subsequent drive bearing from the one or more drive bearings 28, or by a single drive bearing from the one or more drive bearings 28 during a subsequent rotation. The one or more circular platter pumps 154 described may perform similar to peristaltic pumps or roller pumps known in the art. The one or more circular platter pumps 154 are positive displacement pumps for pumping a variety of fluids, and the fluid is contained in a flexible tube fitted inside a circular pump casing. The IV-line 21 leads into the patient, where the fluid being pumped from the fluid solution bag 38 is dispensed. The illustrated embodiment of FIGS. 17 and 18 is shown including two bladder cavities 37 that may each include a fluid solution bag 38. The illustrated embodiment of FIGS. 17 and 18 may include a drive system described herein for each fluid solution bag 38. Said another way, the embodiment illustrated in FIGS. 17 and 18, each may include an IV-line 21 for each fluid solution bag 38 pumped in the same process as described for FIG. 18. The two IV-lines 21 may combine into a single line before entering the patient. Additionally, the pumping rates of each IV-line 21 may be set independently of each other to achieve different mixtures of fluids contained in the two fluid solution bags 38 (shown in FIG. 17).

FIG. 19 illustrates an infusion unit 19 embodiment that includes a stabilizer 200. The stabilizer 200 may comprise or be formed of a breathable, soft, comfortable, and the like material to increase user comfort. For example, the stabilizer may be a textile, woven material, mesh, elastic, etc. The material of stabilizer 200 may be impregnated with anti-microbial and/or anti-bacterial material to reduce transmission of illness between users or visits. Alternatively, stabilizer 200 may comprise or be formed of a sterilizable material, for example a plastic, silicone, or the like. The stabilizer 200 may be coupled to the infusion unit 19 and may stabilize the infusion unit 19. The stabilizer 200 may strap or slide onto the hand of the patient arm 89. The stabilizer 200 may define a thumb aperture 202. The defined thumb aperture 202 may surround the thumb 204 of the patient arm 89. As such, the stabilizer 200 may eliminate roll and translation of the infusion unit 19 to which the stabilizer 200 is coupled. The stabilizer 200 may be used with any infusion unit described herein.

Specific embodiments of an infusion unit have been described herein. It should be apparent, however, to those skilled in the art that many more modifications besides those described are possible without departing from the inventive concepts herein. The inventive subject matter, therefore, is not to be restricted except in the spirit of the appended claims.

The systems and methods of the embodiments and variations described herein can be embodied and/or implemented at least in part as a machine configured to receive a computer-readable medium storing computer-readable instructions. The instructions may be executed by computer-executable components integrated or in communication with the system and one or more portions of the processor on or in communication with the infusion device and/or computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

As used in the description and claims, the singular form “a”, “an” and “the” include both singular and plural references unless the context clearly dictates otherwise. For example, the term “syringe” may include, and is contemplated to include, a plurality of syringes. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An infusion unit comprising:
a foldable housing configured to be secured around an arm of a person,
at least one internal reservoir for temporarily holding a first medication in a bladder;
a connection from the at least one internal reservoir to an intravenous (IV) line;
a dispensing mechanism that forces a medication from within the at least one internal reservoir through the intravenous (IV) line,
wherein the at least one internal reservoir is an IV fluid solution bag that has at least one exit port that connects to the intravenous (IV) line; and
a controller that operates the dispensing mechanism based on a predefined parameter.

2. The infusion unit of claim 1, further comprising at least one sensor that is configured to monitor at least one vital sign of the person.

3. The infusion unit of claim 2, wherein the at least one vital sign is selected from a group consisting of a heart rate, an O2 content, a blood pressure, and an electrocardiogram.

4. The infusion unit of claim 1, further comprising an interface that allows the controller to be programmed externally from the foldable housing.

5. The infusion unit of claim 1, wherein the at least one internal reservoir has an internal volume of about 250 mL or less.

6. The infusion unit of claim 1, wherein the infusion unit comprises at least two internal reservoirs with a second medication in at least one of the at least two internal reservoirs.

7. The infusion unit of claim 6, wherein each of the at least two internal reservoirs have an internal volume of about 5 mL or less.

8. The infusion unit of claim 1, further comprising one or both of a flow sensor or an air bubble sensor.

9. The infusion unit of claim 1, further comprising an outer shell with a hinge and a lock.

10. The infusion unit of claim 1, further comprising a sensor configured to detect a presence of the at least one internal reservoir.

11. The infusion unit of claim 1, further comprising a wireless connection to an external communication device.

12. The infusion unit of claim 1, further comprising a scanner configured to read a label associated with contents of the at least one internal reservoir.

13. The infusion unit of claim 1, further comprising a tamper detection mechanism.

14. A wearable infusion unit comprising:
at least one internal reservoir for temporarily holding a medication in a bladder;
a connection from the at least one internal reservoir to an intravenous (IV) line;
a dispensing mechanism that forces the medication from within the at least one internal reservoir through the intravenous (IV) line;
a controller; and
one or more physiological sensors communicatively coupled to the controller and configured to measure one or more vital statistics of a user wearing the wearable infusion unit,
wherein the controller is configured to receive the one or more measured vital statistics from the one or more physiological sensors.

15. The infusion unit of claim 14, wherein the controller is configured to transmit the one or more measured vital statistics from the one or more physiological sensors to a remote computing device.

16. The infusion unit of claim 15, wherein the controller is configured to receive instructions from the remote computing device based on the one or more measured vital statistics.

17. A method of infusing medication into a patient, the method comprising:
securing an infusion unit onto a forearm of a patient;
fluidly connecting an intravenous (IV) line to a reservoir connected to one or more drive mechanisms of the infusion unit, wherein the one or more drive mechanisms are configured to force medication from the reservoir and into the intravenous (IV) line;
receiving one or more control parameters at a controller communicatively coupled to the infusion unit; and
adjusting, according to the one or more received parameters, the one or more drive mechanisms of the infusion unit to adjust a dispensing of the medication from the reservoir of the infusion unit.

18. The method of claim 17, wherein the one or more control parameters comprise one or more of: a dosing parameter, a volume of the medication, a flow rate of the medication, an activation of the one or more drive mechanisms, or a deactivation of the one or more drive mechanisms.

19. The method of claim 17, further comprising measuring one or more vital statistics of a patient with one or more physiological sensors of the infusion unit.

20. The method of claim 19, further comprising transmitting the one or more measured vital statistics from the infusion unit to a remote computing device.
